(19) United States
(12) Patent Application Publication
(10) Pub. No.: US 2022/0040352 A1
(43) Pub. Date: Feb. 10, 2022

(54) SANITIZING UVC LIGHTING SYSTEM

(71) Applicant: Greg Rosenbaum, Wellington, FL (US)

(72) Inventor: Greg Rosenbaum, Wellington, FL (US)

(21) Appl. No.: 16/984,790

(22) Filed: Aug. 4, 2020

(51) Int. Cl. A61L 2/26; F21V 8/00; A61L 2/10; A61L 2/00; A61L 9/20

(52) U.S. Cl. CPC A61L 2/26; G02B 6/001; G02B 6/0006; A61L 2209/12; A61L 2/0047; A61L 9/20; A61L 2202/11; A61L 2/10

(57) ABSTRACT

A system or kit for distributing UVC light in an environment occupied by humans generally comprises a fiber optic cable, a UVC light assembly that couples to the fiber optic cable and emits a wavelength of 200-250 nm, and a flexible plastic tube that receives and carries the light and distributes the UVC light through its outer surface. UVC light issues from multiple locations along the length of the flexible plastic tube at varied angles toward surfaces in the light's path to disinfect the surfaces and intervening air. The UVC light does not penetrate the outer layer of human skin. One light source may feed multiple flexible plastic tubes, which may have light-emitting perforations and a flat side shaped to aid mounting on many surfaces. An outer finish on the flexible plastic tube may enhance dispersion of the emitted UVC light.

### FIELD

The present disclosure relates to a system, device, and method for distributing UVC light, and more particularly a system, device, and method for sanitizing spaces with UVC light while those spaces are occupied by humans.

### BACKGROUND

This section provides background information related to the present disclosure which is not necessarily prior art.

The general public is increasingly interested in reducing transmission of viruses and other pathogens. Transmission can occur via various routes including person-to-person, aerosolized droplets, and contact with inanimate surfaces.

Disinfection historically has leaned toward room-clearing methods and devices, including manual disinfection and machinery that emits chemicals harmful to humans who are in the vicinity. Such treatment must stop for the room to be re-occupied, then must start again when disinfection is requested, and then continue that cycle.

There is need in the art for a system, device, and method that continuously and adequately disinfects without adverse effect on humans in the vicinity.

### SUMMARY OF THE INVENTION

This section provides a general summary of the disclosure, and is not a comprehensive disclosure of its full scope or all of its features.

A system for distributing UVC light in an environment occupied by humans generally comprises a fiber optic cable, a UVC light assembly that couples to the fiber optic cable and emits UVC light having a wavelength of 200-250 nm along the fiber optic cable, and a flexible plastic tube that receives and carries the UVC light from the fiber optic cable and distributes the UVC light beyond the flexible plastic tube's outer surface. The UVC light issues from multiple locations along the length of the flexible plastic tube to distribute the UVC light at varied angles toward surfaces in the UVC light's path to disinfect the surfaces and intervening air. When distributed into an environment occupied by humans, the UVC light does not penetrate the outer layer of human skin.

The UVC light assembly may have only one light source that is shared by at least two flexible plastic tubes and at least two fiber optic cables coupled to the UVC light assembly, with the plastic tubes running in different directions. Or two fiber optic cables may connect to opposite ends of one flexible plastic tube. The flexible plastic tube may have perforations spaced along its length through which the UVC light emits and may have a flat side shaped to abut a mounting surface. The flexible plastic tube, which may be co-extruded with different UVC-transparency properties, may be at least partially transparent to the UVC light and allow the UVC light to escape along its length.

The fiber optic cable may terminate proximate an open end of the flexible plastic tube and emit light into the flexible plastic tube, and the flexible plastic tube may internally reflect the UVC light along its length. Alternatively, the fiber optic cable is located within and along the length of the flexible plastic tube and may have a cladding that is at least partially transparent to the UVC light, such that the UVC light escapes along the length of the fiber optic cable and is distributed beyond the outer surface of the flexible plastic tube. An outer surface finish on the flexible plastic tube may enhance dispersion of the emitted UVC light.

A kit for distributing UVC light in an environment occupied by humans generally comprises a fiber optic cable, a UVC light assembly that couples to the fiber optic cable and emits UVC light having a wavelength of 200-250 nm along the fiber optic cable, and a flexible plastic tube that receives and carries the UVC light from the fiber optic cable and distributes the UVC light beyond the flexible plastic tube's outer surface, the flexible plastic tube having a flat side shaped to abut a mounting surface. The flexible plastic tube may comprise perforations spaced along its length.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

A more complete understanding of these embodiments, and the attendant advantages and features thereof, will be more readily understood by reference to the following detailed description when considered in conjunction with the accompanying drawings. The drawings described herein may not be to scale, are for illustrative purposes only of selected embodiments and not all possible implementations, and are not intended to limit the scope of the present disclosure.

For clarity and in order to emphasize certain features, not all of the drawings depict all of the features that might be included with the depicted embodiment. The invention also encompasses embodiments that combine features illustrated in multiple different drawings; embodiments that omit, modify, or replace some of the features depicted; and embodiments that include features not illustrated in the drawings. Therefore, it should be understood that there is no restrictive one-to-one correspondence between any given embodiment of the invention and any of the drawings.

FIG. 1 is a front perspective view of a flexible plastic tube.

FIG. 2 is a view of the end or cross-section of FIG. 1.

FIG. 3 is the flexible plastic tube of FIG. 1 with perforations.

FIG. 4 is a cross-section taken through a perforation of FIG. 3.

FIG. 5 is a rear perspective view of FIG. 1.

FIG. 6 is a closed end or end cap of a flexible plastic tube.

FIG. 7 is one or more flexible plastic tubes attached to a mounting surface.

FIG. 8 is chart illustrating a system for distributing far-UVC light.

FIG. 9 is an illustration of light paths through a fiber optic cable.

FIG. 10 is an illustration of multiple fiber optic cables.

FIG. 11 is an illustration of the system of FIG. 8.

FIG. 12 is an illustration of the system of FIG. 8.

FIG. 13 is a flow chart illustrating a method of distributing far-UVC light to disinfect an environment.

Corresponding reference numerals indicate corresponding parts throughout.

### DETAILED DESCRIPTION

Any reference to “invention” within this document is a reference to an embodiment of a family of inventions, with no single embodiment including features that are necessarily included in all embodiments, unless otherwise stated. Furthermore, although there may be references to “improvements” or “advantages” provided by some embodiments, other embodiments may not include those same advantages, or may include different advantages. Any advantages described herein are not to be construed as limiting to any of the claims.

As used herein, relational terms, such as “first” and “second,” “top” and “bottom,” and the like, may be used solely to distinguish one entity or element from another entity or element without necessarily requiring or implying any physical or logical relationship or order between such entities or elements.

Specific quantities, dimensions, spatial characteristics, compositional characteristics and performance characteristics may be used explicitly or implicitly herein, but such specific quantities are presented as examples only and are approximate values unless otherwise indicated. Discussions and depictions pertaining to these, if present, are presented as examples only and do not limit the applicability of other characteristics, unless otherwise indicated.

In describing preferred and alternate embodiments of the technology described herein, specific terminology is employed for the sake of clarity. The technology described herein, however, is not intended to be limited to the specific terminology so selected, and it is to be understood that each specific element includes all technical equivalents that operate in a similar manner to accomplish similar functions.

Example embodiments will now be described more fully with reference to the accompanying drawings. Specific details are set forth such as examples of specific components and methods to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known device structures are not described in detail.

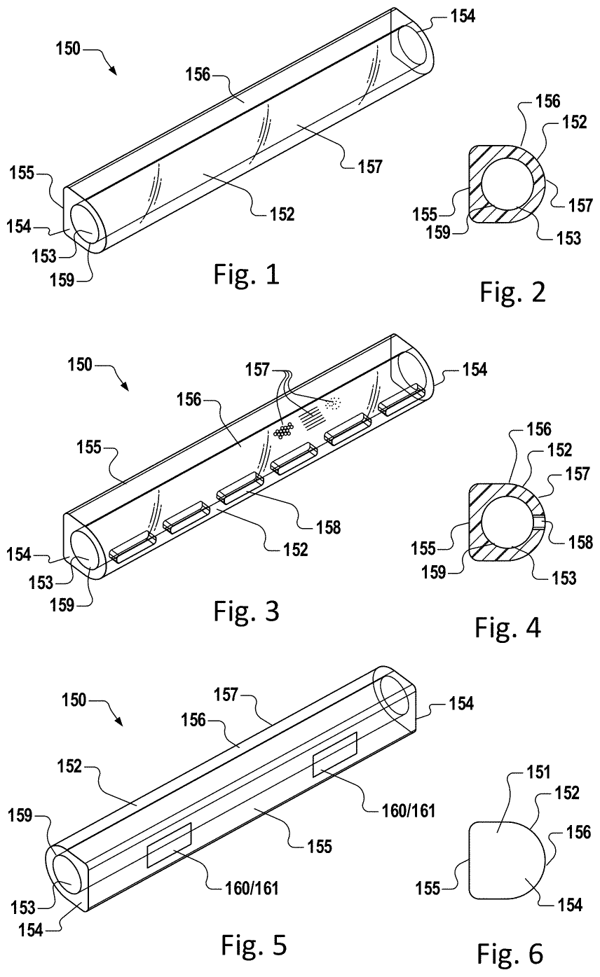

With reference to FIGS. 1-13, a system, device, and method for distributing UVC light provides safe, continuous, low-dose/low-rate exposure of ultraviolet radiation to humans (and other mammals) to inactivate pathogens such as viruses and bacteria before they enter human bodies.

UVC radiation broadly refers to 100-280 nm wavelengths known to be highly germicidal. Existing technologies typically are either very targeted, like surgical devices, or dangerous to humans due to broadcasting of high doses of ultraviolet radiation to sanitize the surrounding environment.

The system of the present Application improves upon existing technology by employing far-UVC light in the wavelength range of 200-250 nm through a flexible plastic tube at a level that is safe for humans over long-term exposure. Further, the system is readily deployable and configurable to everyday environments such as offices and retail stores for line-of-sight disinfection of pathogens. In the remainder of this Description, the terms “far-UVC” and “UVC” are used interchangeably when referring to UVC light in the 200-250 nm range.

With reference to FIGS. 1-12, a system or kit for distributing UVC light 100 generally comprises a light assembly 110, fiber optic cable 130, and flexible plastic tube 150 together employed to sterilize surfaces and air treated with the UVC. Importantly, pathogens may be shielded from exposure if the light does not reach them; therefore, the flexibility of the plastic tube 150 increases the angles at which the light is emitted, thereby increasing line-of-sight to the pathogens.

FIGS. 1-7 illustrate the basic flexible plastic tube 150 that is clear or transparent and has a thickness of about 5-15 mm between an outer surface 152 and inner surface 153. Tube aperture 159 is about 10-15 mm in diameter and typically runs the length of the flexible plastic tube from one end 154 to the opposite end 154 that may have an end cap or closed end 151. Tube 150 and tube aperture 159 are not limited to those dimensions. The cross-section of the aperture 159 is not limited to a circle, and the curvature 156 of the outer surface 152 may differ from the curvature of the aperture 159 and may be asymmetric. In a preferred embodiment, a flat rear surface 155 aids attachment of the flexible plastic tube 150 to various mounting surfaces 180/18; in such a configuration, the flexible plastic tube 150 is D-shaped. An attachment area 160 on rear surface 155 may comprise discrete recesses or a recess that runs the length of the flexible plastic tube 150, as formed by extrusion. Double-sided tape 161 or other adhesive may be used for attachment. Fasteners 162 that wrap around the flexible plastic tube 150 may also be employed, including mechanical and adhesive attachments that are releasable or comparatively permanent.

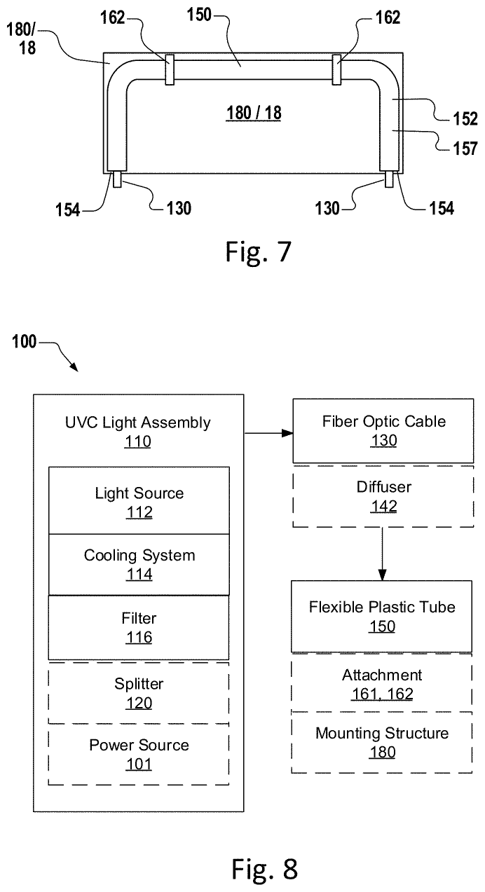

As illustrated in FIG. 7, attachment and flexibility allow one or more tubes 150 to be affixed to a mounting surface 18 of an ordinary structure such as a wall, desk, or ceiling, for example, and to be bent or curved to fit such surface 18. Similarly, one or more tubes 150 may be affixed to a mounting surface 180 of a structure like a partition or stand, for example, that is far-UVC-transparent and designed to allow far-UVC light to treat the environment on both sides of the partition or stand. The UVC light is carried to the flexible plastic tube 150 via one or more fiber optic cables 130 that originate from one or more light assemblies 110, as described later. In addition to the objects already named, mounting surfaces 180 may include, but are not limited to, posts, railings, counters, suspended barriers, barriers on counters, decorative trim, and embedded surfaces in grout. The system 100 or flexible plastic tube 130 may also be added to other products.

A finish 157 may be applied to the outer surface 152 or inner surface 153 to enhance distribution and dispersion of light from the flexible plastic tube 150, whether increasing or decreasing the amount of emitted light as required. Finish 157 may be an applied film, inclusions internal to the plastic, or alteration of the surfaces 152, 153 with dimples, micro-bevels, roughening of the surface, or a combination of any of these, and is not limited to such finishes 157.

Perforations 158 are often added along the length of the flexible plastic tube 150 to allow greater amounts of UVC light to escape, depending upon the composition of the tube material. The perforations 158 are not limited to those shown, but may also be asymmetric and unevenly spaced according to desired functionality.

Combining the transparency of the flexible plastic tube 130 with bends and/or other tubes 130 working in tandem, UVC light issues from multiple locations along the length of the tube(s) 130 to distribute the UVC light at varied angles to sterilize surfaces and intervening air.

Turning now to FIG. 8, UVC light assembly 110 comprises a light source 112 that may be a conventional or specialized lamp or laser, a cooling system 114, at least one filter 116 for modifying or limiting the emitted wavelengths, and a splitter 120 for use with two or more fiber optic cables 130. The short-UVC irradiation range of 200-250 nm wavelength destroys microbes, which are smaller in diameter than human cells. Yet penetration through biological materials (even the outer layer on the surface of human skin, or the outer layer on the outer surface of the eye) is very limited because far-UVC light is strongly absorbed by proteins and other biomolecules (reduced by half within about 0.3 μm of tissue). Damage from repeated, long-term exposure at wavelengths above 250 nm may include inflammation, skin cancer, and damage to lenses in the eyes. Therefore, the present Application utilizes a range well below the ideal medical-grade germicidal wavelength of about 254 nm. Staying within the preferred range of 205-230 nm (ideally 220-225 nm) is seen as an optimum balance of disinfection and safety. Doses as low as 4.5 mJ/cm² have been shown to kill 90% or more of targeted pathogens, and doses from 75-450 mJ/cm² are highly effective. (Ultraviolet dosage=Ultraviolet intensity x Exposure time) Research indicates a potential compounding benefit from short exposures over time, meaning that the benefit builds over time as the same area or object is repeatedly treated, and a dose of about 150 mJ/cm² has been shown to be both highly effective and safe for human skin with long-term exposure.

Preferred light assemblies 110 include, but are not limited to, a Krypton-bromine (Kr—Br) excimer lamp, a Krypton-chloide (Kr—Cl) excimer lamp having maximum output wavelength of 222 nm, and a Helium-Argon (HE-AG) laser having a wavelength of 224 nm. One of skill in the art will understand that the far-UVC light assembly need not be explained in detail here, but may include filters, fans or other cooling systems, mirrors, sensors, etc. Parts shown or not shown may be varied to keep the power rate stable and control the wavelength range to achieve the total system 100 functionality intended herein. Likewise with the fiber optic cables 130.

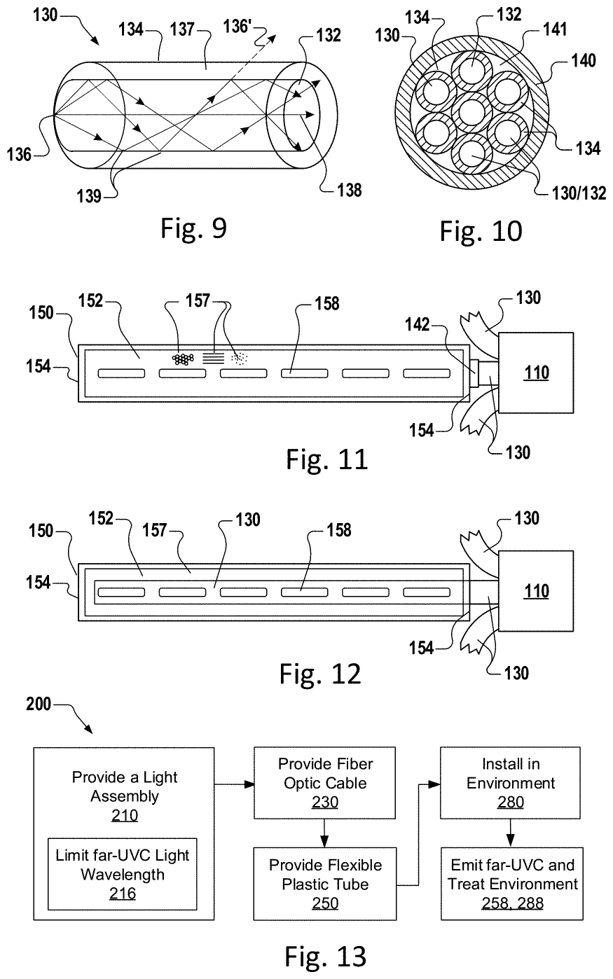

Fiber optic cables 130 of FIGS. 9-12 typically comprise a glass core 132 with a plastic jacket or cladding 134. Light beam path 136 may be single-mode 138 or multi-mode 139 and engineered with a reflective index in mind to optimize use of the light carried along the core 132. Multiple fiber optic cables 130 may be gathered and secured by an outer jacket 140 along with additional coatings 141 and/or cladding for use in certain applications of the present system 100. In one configuration, the fiber optic cable's 130 plastic cladding 140, 134 may be far-UVC-transparent in order to allow far-UVC light to escape 136′ through the cladding 134 (and outer jacket 140, if used). Degrees of far-UVC transparency may be designed as desired for specific functionality.

One of skill in the art will understand that the terms “transparent” and “clear” are often used interchangeably to refer to plastics that are “see-through,” unless one specifies that “clear” means “totally transparent.” With reference to far-UVC light, the term “transparent” has an additional meaning in that certain plastics let more far-UVC pass through, making them more transparent to the light even though they might not look more transparent to the human eye. Acrylic, UV polyethylene, and most other plastics block UVC light with wavelengths less than 300 nm. Other polyethylene is commonly produced that allows far-UVC light to pass through.

FIGS. 11-12 illustrate light assembly 110 comprising one or more fiber optic cables 130, one of which directs light to flexible plastic tube 150. A splitter (not shown) is employed for use with multiple fiber optic cables 130 that are typically oriented in different directions. For example, one cable 130 may lead to a wall, and another cable 130 may lead to a desktop. Those two cables 130 and their respective “light registers” or flexible plastic tubes 150 may treat different areas of a room, or one may shine down while the other shines up, thus reducing shading and sheltering of pathogens. Multiple fiber optic cables 130 sharing one light source 112 also decreases the relative cost of the light assembly 110 per area treated.

In FIG. 11, the fiber optic cable 130 leads to a diffuser 142 that helps spread the UVC light down the length of flexible plastic tube 150. In this embodiment, the flexible plastic tube 150 is constructed of material that keeps a substantial amount of light within the tube 150 to ensure that light emits along the tube's 150 length. The surface finish 157 and perforations 158 enhance management of the volume of light that escapes the tube's outer surface 152. The flexible plastic tube 150 may be a single extruded material or may be a co-extrusion having different but cooperating physical properties. For example, portions of the flexible plastic tube 150 may have low far-UVC transparency to keep light inside, which is beneficial around bends in the tube 150, and other portions of the tube 150 may have high far-UVC transparency to allow the light to escape toward environments needing disinfection. This configuration is not limited to use of a diffuser 142. FIG. 12 differs in that an intubated fiber optic cable 130 runs the length of the sheath or flexible plastic tube 150. If that fiber optic cable 130 has a transparent cladding 134, then the tube 150 may also have a more far-UVC-transparent structure due to the fiber optic cable 130 carrying the light down the flexible plastic tube 150. The cladding 134 may be the flexible plastic tube 150.

Referring now to FIG. 13, in practice, a light assembly is provided 210 and limited 216 to a range of far-UVC wavelengths from 200-250 nm. A power source 101 may or may not be considered part of the light assembly 110 or system 100. One or more fiber optic cables are provided 230 and matched with flexible plastic tube(s) 250, then installed in the targeted environment 280. The tube emits far-UVC light through its outer surface 258 and treats the environment 288 without harming human occupants.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for distributing UVC light in an environment occupied by humans, the system comprising:
(a) a fiber optic cable;
(b) a UVC light assembly that couples to the fiber optic cable and emits UVC light having a wavelength of 200-250 nm along the fiber optic cable; and
(c) a flexible plastic tube that receives the UVC light from the fiber optic cable and distributes the UVC light beyond the flexible plastic tube's outer surface;
wherein the UVC light issues from multiple locations along the length of the flexible plastic tube to distribute the UVC light at varied angles toward surfaces in the UVC light's path to disinfect the surfaces and intervening air.

2. The system of claim 1, wherein the wavelength is 220-225 nm and, when distributed into an environment occupied by humans, the UVC light does not penetrate the outer layer of human skin.

3. The system of claim 1, comprising at least two flexible plastic tubes and at least two fiber optic cables coupled to the UVC light assembly, wherein a first fiber optic cable connects to a first flexible plastic tube oriented in a first direction and a second fiber optic cable connects to a second flexible plastic tube oriented in a second direction, and wherein the UVC light assembly has only one light source.

4. The system of claim 1, comprising at least two fiber optic cables coupled to the UVC light assembly, wherein the two fiber optic cables connect to opposite ends of the flexible plastic tube.

5. The system of claim 1, the flexible plastic tube having a flat side shaped to abut a mounting surface.

6. The system of claim 1, wherein the fiber optic cable terminates proximate an open end of the flexible plastic tube and emits light into the flexible plastic tube.

7. The system of claim 1, wherein the flexible plastic tube internally reflects the UVC light along its length.

8. The system of claim 1, the flexible plastic tube comprising perforations spaced along its length through which the UVC light emits.

9. The system of claim 1, wherein the flexible plastic tube is at least partially transparent to the UVC light and allows the UVC light to escape along the length of the flexible plastic tube.

10. The system of claim 1, wherein the fiber optic cable is located within and along the length of the flexible plastic tube and has a cladding that is at least partially transparent to the UVC light, and wherein the UVC light escapes along the length of the fiber optic cable and is distributed beyond the outer surface of the flexible plastic tube.

11. The system of claim 1, the outer surface of the flexible plastic tube comprising a surface finish that enhances dispersion of the emitted UVC light.

12. The system of claim 1, the flexible plastic tube comprising co-extrusion of two plastics having different UVC light transmitting properties.

13. A system for distributing UVC light in an environment occupied by humans, the system comprising:
(a) at least two fiber optic cables;
(b) a UVC light assembly that consists of one light source and couples to the at least two fiber optic cables and emits UVC light having a wavelength of 200-250 nm along the at least two fiber optic cables; and
(c) at least two flexible plastic tubes that receive the UVC light from the at least two fiber optic cables and distributes the UVC light beyond the at least two flexible plastic tubes' outer surfaces;
wherein the UVC light issues from multiple locations along the lengths of the at least two flexible plastic tubes to distribute the UVC light at varied angles toward surfaces in the UVC light's path to disinfect the surfaces and intervening air.

14. The system of claim 13, the at least two flexible plastic tubes having a flat side shaped to abut one or more mounting surfaces.

15. The system of claim 13, wherein the at least two flexible plastic tubes internally reflect the UVC light along their lengths.

16. The system of claim 13, the at least two flexible plastic tubes comprising perforations spaced along their lengths through which the UVC light emits.

17. The system of claim 13, wherein the at least two flexible plastic tubes are at least partially transparent to the UVC light and allow the UVC light to escape along the lengths of the at least two flexible plastic tube.

18. The system of claim 13, the at least two flexible plastic tubes comprising co-extrusion of two plastics having different UVC light transmitting properties.

19. A kit for distributing UVC light in an environment occupied by humans, the kit comprising:
(a) a fiber optic cable;
(b) a UVC light assembly that couples to the fiber optic cable and emits UVC light having a wavelength of 200-250 nm along the fiber optic cable; and
(c) a flexible plastic tube that receives the UVC light from the fiber optic cable and distributes the UVC light beyond the flexible plastic tube's outer surface, the flexible plastic tube having a flat side shaped to abut a mounting surface;
wherein the UVC light issues from multiple locations along the length of the flexible plastic tube to distribute the UVC light at varied angles toward surfaces in the UVC light's path to disinfect the surfaces and intervening air.

20. The kit of claim 19, the flexible plastic tube comprising perforations spaced along its length through which the UVC light emits.
